(19) United States
(12) Patent Application Publication
(10) Pub. No.: US 2024/0341986 A1
(43) Pub. Date: Oct. 17, 2024

(54) DELIVERY SYSTEM FOR A FURCATED STENT

(71) Applicant: TRISTENT DEVELOPMENT SAS, Annecy (FR)

(72) Inventors: Omer Faruk EKER, Lyon (FR); Kamil Jerzy CHODZYNSKI, Mons (BE)

(21) Appl. No.: 18/626,056

(22) Filed: Apr. 3, 2024

Related U.S. Application Data
(63) Continuation-in-part of application No. 17/564,044, filed on Dec. 28, 2021, now Pat. No. 11,974,933.
Application No. 17/564,044 is a continuation of application No. 16/478,923, filed on Jul. 18, 2019, now Pat. No. 11,229,538.

(30) Foreign Application Priority Data
Jan. 19, 2017 (EP) 17290006.0
Jan. 25, 2017 (BE) 20175043

(51) Int. Cl. A61F 2/954; A61F 2/06; A61F 2/07; A61F 2/90; A61F 2/95; A61F 2/966; A61F 2/97

(52) U.S. Cl. CPC A61F 2/954; A61F 2/07; A61F 2/90; A61F 2/97; A61F 2002/065; A61F 2/9522; A61F 2/966; A61F 2002/9665; A61F 2210/0014; A61F 2250/0067

(57) ABSTRACT

A delivery system for a furcated stent having a stem and at least two arms, the delivery system including a delivery catheter comprising an elongated first tube having a proximal end and a distal end, and a tubular furcated part at the distal end configured to accommodate the arms; wherein the tubular furcated part includes two or more limbs, each limb provided with a slit, thereby providing multiple limbs having multiple slits, the multiple slits together form a passageway for passage therethrough of the furcated stent, the passageway has a narrowed state and a widened state and is biased in the narrowed state, and the tubular furcated part is formed of a compliant material and is configured to allow repeatable transition between the passageway narrowed state and passageway widened state.

### RELATED APPLICATION

This application is a continuation-in-part (CIP) of U.S. patent application Ser. No. 17/564,044, filed Dec. 28, 2021, which is a continuation of U.S. patent application Ser. No. 16/478,923, filed Jul. 18, 2019 (U.S. Pat. No. 11,229,538), which is a U.S. National Phase Application of PCT/EP2018/050554, filed Jan. 10, 2018, and which claims priority to European Patent Application EP 17290006.0, filed Jan. 19, 2017, and Belgium patent application BE20175043, filed Jan. 25, 2017, all of these applications are incorporated by reference in their entireties.

### TECHNICAL FIELD OF THE INVENTION

Delivery system for a furcated stent and method for treating a bodily vessel (e.g. aneurysm or stenosis or occlusion of the vessel) located at the furcation of the bodily vessel are described herein. The system and method may be implemented for furcation aneurysms or occlusions located intracranially or extracranially (i.e., aortic or peripheral aneurysms).

### BACKGROUND TO THE INVENTION

An intracranial aneurysm is a saccular, or more rarely a fusiform, dilatation of a cerebral artery due histological structural changes in the arterial wall. It is responsible for a wall weakness which is at risk of rupture if untreated and hence of intracranial bleeding (subarachnoid haemorrhage and/or intra parenchymal). Amongst younger populations, intracranial aneurysmal disease represents the most prevalent predisposition to a fatal risk with an estimated incidence of 5-7 cases per 100,000 persons per year and prevalence between 2-5%. It is the leading cause of haemorrhagic stroke and is responsible for a reduction in life expectancy and for potentially severe disabilities affecting quality of life. The mortality rate associated with subarachnoid haemorrhage secondary to ruptured intracranial aneurysm is estimated at 45-50% at thirty days. The associated morbidity is estimated at 25-30%, with dependence in nearly 30% of patients at one year despite appropriate and early treatment. The risk of rupture of unruptured intracranial aneurysm is difficult to estimate and varies between 0.4% and 17.8% at five years. It is increased by the following factors: age, gender, hypertension, smoking, aneurysm size and location in posterior circulation. Finally, the aneurysmal rupture and its complications have a significant economic impact in terms of costs related to the management of the acute phase, the neurological deficits and/or subsequent dependence. In the Europe alone, thirty-five to thirty-seven million people may have intracranial aneurysms. In France alone, six thousand intracranial haemorrhages secondary to ruptured intracranial aneurysms occur each year.

The main goal of intracranial aneurysm treatment, whatever the chosen method, is to exclude the malformative out-pouching from the blood circulation. Most of the current practices for treating intracranial aneurysms are based on endovascular catheter-based approaches mainly represented by the aneurysm's filling with a filler material (the so-called “coiling” technique with platinum coils and/or cellulose acetate polymer). The filling material deployment within the aneurysm may be associated to the inflation of a balloon within the arterial lumen during the filling material deployment (called the “balloon remodeling method”) or to the deployment of stent within the artery covering aneurysm's neck (called the “stent assisted coiling method”) in order to prevent its protrusion within the artery. It results in a thrombus formation inside the aneurysm and its exclusion from the blood circulation. These techniques have become very quickly the gold standard methods for intracranial treatment. The historical surgical clipping approach consists in the placement of an aneurysm clip across the aneurysm to prevent blood flow into the aneurysm. This technique is more invasive and less and less carried out because of its high risk, especially for elderly or medical complicated patients.

The described methods above are related to remain device permanently in the body for aneurysm treatment or related to its transitory deployment in the arterial furcation lumen and then its retrieval for the treatment or arterial occlusion. For the first purpose, the latest invention of biodegradable polymers may solve this problem and device will be able to be removed from the body by dissolving biopolymers itself by time or using chemicals components. For the last purpose, the stent may be deployed and remain permanently in the body in case of arterial stenosis and therefore it presents the same material characteristics described above apart from its design (laser cut instead of braided mesh).

Despite the significant advance allowed and the paradigm shift created in the management of intracranial aneurysms by endovascular techniques, furcation aneurysms and especially those presenting a large neck or a neck encompassing one or both of the division branches remain challenging for their treatment. Stenting techniques represent an alternative for those aneurysms. Presently, many strategies are employed to treat intracranial furcation aneurysms with currently available stent designs (either laser cut or braided stents) including: stent assisted coiling, Y-stent, T-Stent or Crush-stent techniques. With stents, one common approach is to place the stent (either laser cut or braided stent) in the main artery and one of its branch (more commonly the biggest branch or the branch more affected by the aneurysm) before (jailing method) or after coiling the aneurysm sac. If the first deployed stent is not efficient enough to prevent the coil protrusion into the arterial lumen, a second stent is deployed within the first stent and the second branch (called Y-stent technique). This latter technique may be used as first option according to the arterial anatomy and the access difficulties. However, with these methods the amount of material within the arterial lumen and precisely across the arterial branche(s) are source of thromboembolic complications, increased in Y-stent, T-Stent and Crush-stent techniques. Recently, flow diversion technique based on the deployment of flow diverter stent within the parent artery and flow disruption technique based on the deployment of an intrasaccular flow disruption device within the aneurysm became also options for those challenging aneurysms. Because of the higher amount of material in their design, the flow diverter stents are at higher risk of thromboembolic complications. Additionally, in arterial furcation the deployed flow diverter stent within the arterial lumen covers not only the side perforators but also the division branch which may compromise its patency at long term. The flow disruption device method consists of a frame, based on the same braided meshes as braided stents, that is deployed into the aneurysm sac in

order to fill in it, thereafter to stop the flow within the aneurysm sac and to promote the intrasaccular thrombosis. This approach is limited either by the incomplete occlusion of the aneurysm at its neck or the bulging of the frame into the arterial lumen that imposes a secondary rescue stenting. These limitations are due to the design of the frame which in most of cases despite its compliance does not enable the perfect conformation of the device to the aneurysm neck and wall.

### SUMMARY

The invention disclosed herein may be deployed to overcome problems encountered in the art. It is a further aim to provide new therapies for certain type of intracranial aneurysms located at the furcation of an artery for which the present day therapies are widely regarded as inadequate. The invention may be deployed to provide treatments for certain type of dysfunction located at the furcation of any bodily vessel.

Disclosed herein is a delivery system for a furcated stent having a stem and at least two arms, the delivery system comprising: a delivery catheter comprising an elongated first tube having a proximal end and a distal end, and a tubular furcated part at the distal end configured to accommodate the arms; wherein: tubular furcated part includes two or more limbs, each limb provided with a slit, thereby providing multiple limbs having multiple slits; the multiple slits together form a passageway for passage therethrough of the furcated stent; the passageway has a narrowed state and a widened state, is biased in the narrowed state, and is configured for repeatable transition between the narrowed state and the widened state.

A quantity of arms of the furcated stent may be 2, 3, 4, 5 or 6, and wherein a quantity of limbs of the tubular furcated part is 2, 3, 4, 5 or 6 respectively (and a quantity of arms of the furcated stent may be equal to or less than a quantity of limbs of the elongated first tube (102) of the delivery catheter).

The repeatable transition between the narrowed state and widened state of the passageway may be actuated by application and/or withdrawal of mechanical force between the passageway and the furcated stent.

The tubular furcated part may be configured to constrain radially the arms when the passageway in the narrowed state and resist expansion of the furcated stent when the passageway in the narrowed state.

Each slit may extend from each limb in a proximal direction from a distal terminal end of the limb, and connects at its proximal end to at least one other slit to form the passageway.

The slit has edges that are always detached from each other.

The multiple limbs may be joined at a joint and spread apart as the limbs extend in a distal direction.

The furcated stent may be self-expanding or non-self-expanding.

The tubular furcated part may be formed of a compliant material that provides the bias of the passageway in the narrowed state and the repeatable transition between the narrowed state and widened state.

The delivery system as described herein may further contain: an access catheter including an elongated second tube having a proximal end region and a distal end region provided with a second lumen adapted to slidably accommodate the first tube, and configured to control a gradual opening or folding of the tubular furcated part of the first tube responsive to slidable relative displacement of the first and second tubes.

The delivery catheter may be configured for an over-the-wire or rapid exchange mode of operation.

The delivery system may further comprise the furcated stent that is self-expanding or non-self-expanding.

The furcated stent may be provided with an elutable active pharmaceutical ingredient.

The furcated stent may be prepared by laser cutting or braiding.

The delivery system may further comprise a pusher including an elongated flexible rod having a proximal end region and a distal end region, and a capture element at the distal end region of the elongated flexible rod for releasable attachment to the furcated stent at the proximal end region which and wherein the capture element is radially self-expanding to adopt an open or folded configuration, wherein the folded configuration is configured for passage within a first lumen of the first tube and peripheral edges of the capture element are closer together to grip a proximal end of the furcated stent, and wherein the open configuration is configured for release of the furcated stent.

The delivery system may further comprise a loader for loading of the furcated stent into the delivery catheter comprising an elongated third tube having a proximal end and a distal end provided with a third lumen adapted to slidably accommodate the furcated stent in a folded configuration, wherein the distal end of the third tube is configured to couple with a proximal end of the delivery catheter such that a first lumen of the delivery catheter and the third lumen are connected to form a continuous passage for advancement of the furcated stent in the folded configuration from the loader to the delivery catheter.

The delivery catheter may be for a bodily vessel.

The bodily vessel may be an artery, a vein, a trachea-bronchial tree, a pancreaticobiliary duct, a urinary tract, or salivary excretory duct.

Further provided is a kit comprising the delivery catheter as defined in claim 1, further comprising at least one of: an access catheter, a pusher, a loader, or at least one guidewire.

Further provided is a delivery assembly comprising: a furcated stent including a stem and first arms; a delivery catheter comprising an elongated first tube having a proximal end, and a distal end, and a tubular furcated part proximate the distal end of the first tube, wherein the tubular furcated part includes second tubular arms configured to receive within the second tubular arms the first arms of the furcated stent; and each second tubular arm includes a slit extending substantially the length of the tubular second tubular arm, each slit contacting at least one other slit thereby forming a passageway, wherein the tubular furcated part is formed of a material having a native state that both applies a bias to the furcated stent resisting expansion of the furcated stent and allows forward and reverse passage of the furcated stent through the passageway, wherein the passageway has: a narrowed state during which the second tubular arms extend circumferentially around a majority of the circumference of each of the first arms, and wherein the second tubular arms are biased in the narrowed state, and a widened state formed by first arms forcing the passageway apart and the second arms extending circumferentially each of the first arms less than in the narrowed state.

The slit edges are always detached from each other.

The second tubular arms may be joined at a joint and spread apart as the second tubular arms extend away from the distal end of the first tube.

Each slit may extend through the joint and along entire lengths of the second tubular arms.

The delivery catheter may be for a bodily vessel.

The delivery catheter may be for a vascular vessel, an artery, a vein, a trachea-bronchial tree, a pancreaticobiliary duct, a urinary tract, or salivary excretory duct.

A method is disclosed herein for delivery of a furcated stent to a site of treatment using a delivery system comprising: advancing a delivery catheter through a passage of a bodily vessel to a treatment site, wherein the delivery catheter includes an elongated first tube having a tubular furcated part at a distal end region of the elongated tube, and the tubular furcated part includes tubular lumens each with a slit extending substantially the length of the tubular lumen forming a passageway, and the tubular lumens are formed of a compliant material configured to allow the slit to expand; and deploying the furcated stent at the treatment site by withdrawing the delivery catheter to advance arms of the furcated stent each through a respective one of the tubular lumens to cause the slit to expand and allow the furcated stent to separate from the tubular lumens, wherein the passageway has: a narrowed state during which each of the tubular lumens extends circumferentially around a majority of a circumference of a respective arm of the arms of the furcated stent, and wherein the tubular lumens are biased in the narrowed state; and a widened state formed by the arms of the furcated stent forcing the passageway apart, and each tubular lumen extends circumferentially around a circumference of a respective arm of the arms of the furcated stent less than in the narrowed state.

The method may further comprise treating the bodily vessel with the furcated stent deployed at the treatment site.

The bodily vessel may be an artery and the treating of the bodily vessel includes treatment of an arterial aneurysm or arterial occlusion or arterial narrowing, or any pathological process that affects or involves the vessel wall or lumen.

Further provided is a delivery system for a furcated stent having a stem and at least two arms, the delivery system comprising: a delivery catheter comprising an elongated first tube having a proximal end and a distal end, and a tubular furcated part at the distal end configured to accommodate and radially constrain the arms; wherein the tubular furcated part includes tubular lumens; wherein a longitudinal slit is disposed on each of the tubular lumens, and the longitudinal slits together form a passageway configured for releasable passage of the furcated stent therethrough; wherein each of the slit edges is apart or touching; wherein each of the slits spans no more than 30% of a circumference of each of the lumens while the slit is closed; and wherein the tubular furcated part is formed of a compliant material configured to resist expansion of the furcated stent, and allow repeatable narrowing and widening of the passageway.

Further provided is a delivery system for a furcated stent having a stem and at least two arms, the delivery system including a delivery catheter comprising: an elongated first tube having a proximal end region and a distal end region, and a tubular furcated part at the distal end region configured to accommodate the arms, wherein the tubular bifurcated part includes tubular lumens; and a passageway formed from a plurality of connected longitudinal slits, each (one) longitudinal slit being disposed on each of the tubular lumens; wherein the tubular furcated part is formed of a material having a native state that resists expansion of each of the arms of the furcated stent while in a respective one of the tubular lumens and allows forward and reverse passage of the furcated stent through the passageway; and

wherein the passageway has: a narrowed state during which each of the tubular lumens extending circumferentially around a majority of a circumference of a respective arm of the at least two arms; and a widened state formed by the at least two arms forcing the passageway apart and each of the tubular lumens extending circumferentially around less than the majority of the circumference of the respective arm of the at least two arms.

Described herein is a delivery system (100) for a bifurcated stent (200) having a stem (226) and a pair of arms (220, 222), comprising a delivery catheter (110) comprising an elongated first tube (102) having a proximal end (20) and a distal end (30) and a bifurcated part (114) at the distal end (30) configured to accommodate the arms (220, 222), wherein a longitudinal slit (140) disposed on the bifurcated part (114) is configured for releasable passage of the bifurcated stent (200) therethrough.

The longitudinal slit may extend from a first limb (120) to a second limb (122) of the bifurcated part (114).

The bifurcated part (114) of the first tube (102) may comprise a first (120) and second (122) limb each configured for passage through a branch (422, 424) of a bifurcated bodily vessel.

The first (120) and second (122) limbs may each be configured to compress radially the bifurcated stent (200).

The delivery system (100) may further comprise an access catheter (300) comprising an elongated second tube (302) having a proximal end (20) and a distal end (30) provided with a second lumen (330) adapted to slidably accommodate the first tube (102), and configured to control a gradual opening or folding of the bifurcated part (114) of the first tube (102) responsive to slidable relative displacement of the first (102) and second (302) tubes.

The delivery catheter (110) may be configured for an over-the-wire or rapid exchange mode of operation.

The delivery system (100) may further comprise the bifurcated stent (200) that is self-expanding. The bifurcated stent (200) may be provided with an elutable active pharmaceutical ingredient, optionally having an antithrombotic property, or anticoagulant property, or endothelisation property, or that is a stimulator of cell migration, or a stimulator of cell growth. The bifurcated stent (200) may be prepared by laser cutting or braiding.

The delivery system (100) may further comprise a pusher (500) comprising an elongated flexible rod (510) having a proximal (20) and distal (30) end, and a capture element (520) at the distal (30) end for releasable attachment to the bifurcated stent (200) at its proximal end (20) which capture element (520) is radially self-expanding to adopt an open or folded configuration, wherein the folded configuration is configured for passage within the first lumen (230) of the first tube (102) and the peripheral edges of the capture element (520) are closer together to grip the proximal end (20) of the bifurcated stent (200), and wherein the open configuration is configured for release of the bifurcated stent (200).

The delivery system (100) may further comprise a loader (600) for loading of the bifurcated stent (200) into the delivery catheter (110) comprising an elongated third tube (602) having a proximal end (20) and a distal end (30) provided with a third lumen (630) adapted to slidably accommodate the bifurcated stent (200) in the folded configuration, wherein the distal end (30) of the third tube (602) is configured to couple with the proximal terminal end of the delivery catheter (110) such that the first (130) and third (630) lumens are connected to form a continuous passage for advancement of the bifurcated stent (200) in the folded state from the loader (600) to the delivery catheter (110).

Described herein is a kit comprising the delivery catheter (110) as defined herein, and one or more of: an access catheter (300) as defined herein, a bifurcated stent (200) as defined herein, a pusher (500) as defined herein, a loader (600) as defined herein, or one or more guidewires such as two, three, or four guidewires such as no more than six guidewires.

Described herein is a method for delivery of a bifurcated stent to a site of treatment using the delivery system (100) as defined herein comprising the steps:
advancing intravascularly the delivery catheter (110) loaded with the bifurcated stent (200) to the site of treatment through the access catheter (300),
opening gradually the delivery catheter (110) by withdrawal of the access catheter (300), and
deploying the bifurcated stent (200) through the slit (140) by withdrawal of the delivery catheter (110).

The treatment may be of an arterial aneurism or arterial occlusion.

### SUMMARY OF DRAWINGS

FIG. 1 is a schematic view of a delivery catheter with bifurcated first tube as described herein.

FIG. 1A show a transverse cross-sectional view of FIG. 1 through main part at plane A.

FIGS. 1B-1 and 1B′-1 show a transverse cross-sectional view of FIG. 1 through planes B and B′ respectively wherein the arms have a slit with abutting edges.

FIGS. 1B-2 and B′-2 show a transverse cross-sectional view of FIG. 1 through planes B and B′ respectively wherein the arms have a slit with overlapping edges.

FIG. 2 is a schematic view of a furcated stent as described herein that is a bifurcated stent.

FIGS. 3A to 3C show a sequence of gradual unfolding (opening) the delivery catheter by slidable actuation of the access catheter.

FIGS. 4A to 4D show a sequence of delivering and unfolding (opening) the delivery catheter at the site of treatment, and deployment of the furcated stent that is a bifurcated stent.

FIG. 5 depicts a pusher having a capture element in an open configuration.

FIG. 6 depicts the pusher of FIG. 5 in abutting alignment with a furcated stent that is a bifurcated stent.

FIGS. 7A to 7C depict a loading sequence for loading a furcated stent that is a bifurcated stent into the limbs of a delivery catheter.

FIGS. 8A to 8E depict a loading sequence for loading a furcated stent that is a bifurcated stent a first lumen of delivery catheter.

FIGS. 9A to 9B depict a sequence of deployment of the furcated stent that is a bifurcated stent using the pusher.

FIG. 10 depicts different bifurcated stent configurations A to H.

FIG. 11 is a schematic view of a delivery catheter with trifurcated first tube as described herein.

FIG. 11A illustrates a view of the delivery catheter of FIG. 11 in a distal to proximal direction.

FIG. 12 is a schematic view of a delivery catheter with quadfurcated first tube as described herein.

FIG. 12A illustrates a view of the delivery catheter of FIG. 12 in a distal (30) to proximal direction.

### DETAILED DESCRIPTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” where used herein when referring to a measurable value such as a parameter, an amount, a temporal duration, and the like, is meant to encompass variations of +/−10% or less, such as +/−5% or less, or +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one” where used herein, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. It is to be understood that other embodiments may be utilised and structural or logical changes may be made without departing from the scope of the present invention. The following detailed description, therefore, is not to be taken in a limiting sense, and the scope of the present invention is defined by the appended claims.

The terms “distal”, “distally” or “distal to” and “proximal”, “proximally” or “proximal to” are used throughout the specification, and are terms generally understood in the field to mean towards (proximal) or away (distal) from the surgeon's side of the apparatus. Thus, “proximal”, “proximally” or “proximal to” means towards the surgeon's side and, therefore, away from the patient's side. Conversely, “distal”, “distally” or “distal to” means towards the patient's side and, therefore, away from the surgeon's side.

The term “furcation” or “furcated” is known in the art and refers to a division or forking of a structure into two or more branches. Where there are two branches or forks is it known as bifurcation or bifurcated. Where there are three branches or forks is it known as trifurcation or trifurcated. Where there are four branches or forks is it known as quadfurcation or quadfurcated, etc. The point or region where the structure branches or forks is known as a furcation point. As described herein, a furcated stent branches or forks into two or more arms. As described herein, an elongated first tube of a delivery catheter branches or forks into two or more limbs or tubular lumens. As described herein, a bodily vessel branches or forks into two or more division branches.

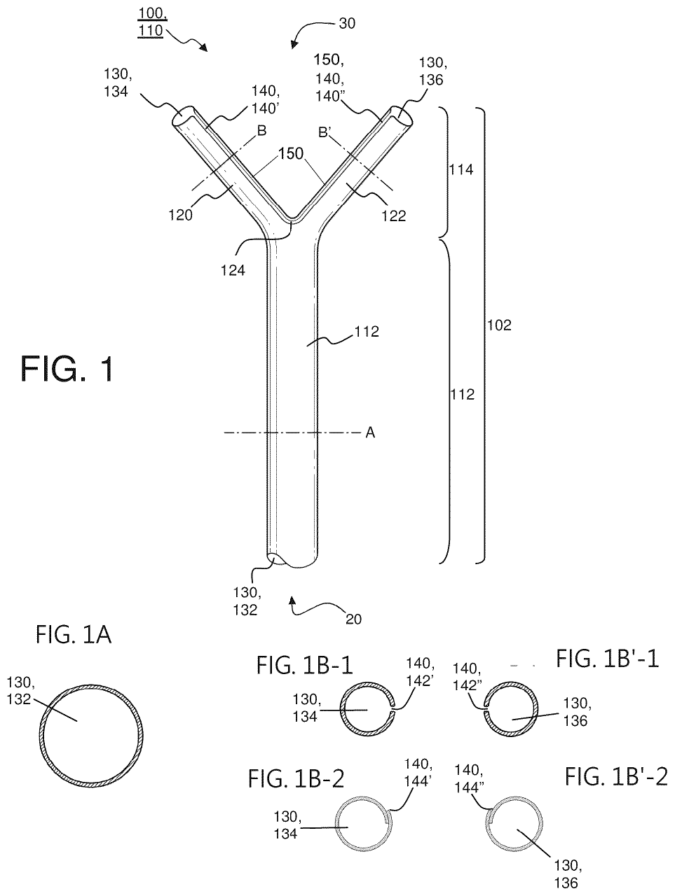

A first aspect relates to a delivery system (100) for a furcated stent (200) having a stem (226) and at least two arms (220, 222) comprising: a delivery catheter (110) comprising an elongated first tube (102) having a proximal end (20) and a distal end (30) and a furcated part (114) at the distal end (30) configured to accommodate the arms (220, 222), wherein a longitudinal slit (140) disposed on the furcated part (114) is configured for releasable passage of the furcated stent (200) therethrough. An exemplary delivery catheter (110) is shown in FIGS. 1, 11 and 12.

The delivery system (100) is used in the treatment of a bodily vessel, in particular of a vascular vessel such as an artery.

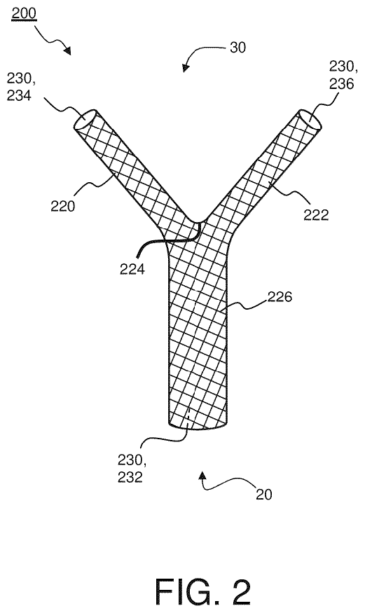

The furcated stent (200) is a stent having a proximal end (20) and a distal end (30), comprising a stem part (226) at the proximal end (20) furcating into two or more arms (220, 222) at the distal end, as illustrated, for instance, in FIG. 2 showing a stent having two arms (bifurcated). A stent lumen (230) extends from the proximal end (232) of the stem to the distal end of each arm (234, 236). The ends of the furcated stent (200) may be open for the passage of bodily fluid in situ. The furcated stent (200) is flexible and may be compliant.

The furcated stent (200) may be used for the treatment of a bodily vessel having a wall and a furcation into two or more division branches.

The bodily vessel may be any time of bodily vessel having a furcation, such as, for instance, an artery, a vein, a trachea-bronchial tree, a pancreaticobiliary duct, a urinary tract, salivary excretory duct.

The bodily vessel for treatment is a fluid conducting vessel that furcates into 2 or more division branches, typically no more than 6 branches. The bodily vessel may be bifurcated (one pair of division branches), or trifurcated (three division branches), or quadfurcated (four division branches). It is understood the bodily vessel may contain more than four division branches (e.g. pentafurcated (five division branches), hexafurcated (six division branches)).

The furcated stent (200) may be used for the treatment of any dysfunction of, or any pathological process that affect or involve the vessel wall or lumen. The treatment and/or dysfunction is at the location of the vessel wall or lumen where the furcated stent is implanted.

The furcated stent (200) may be used for the treatment of an aneurysm located at the furcation of the bodily vessel. The aneurysm having a neck may be located at the vessel furcation communicating with the bodily vessel lumen at the furcation, and communicating or not with predominantly one or both division branches. The aneurysm may encompass one or both the division branches.

The furcated stent (200) may be used for the treatment of a narrowing or an occlusion to vessel wall. The occlusion may encompass one, two, three, or all division branches.

According to one aspect the occlusive material (e.g. clot, thrombus, embolus, stenosis) is penetrated by an arm of the furcated stent (200), and the furcated stent (200) is withdrawn thereby removing at least part of the occlusive material from the site of treatment.

According to another aspect the occlusion is opened by an inflatable balloon. The opening may be prior to deployment of the furcated stent (200), or may be simultaneously with expansion of the furcated stent (200). Where it is simultaneous, the delivery catheter may accommodate an additional tube or lumen for inflation of the inflatable balloon; and the furcated stent (200) balloon-expandable (non-self expanding).

The furcated stent may be bifurcated (one pair of arms), or trifurcated (three arms), or quadfurcated (four arms). It is understood the furcated stent may contain more than four furcations (e.g. pentafurcated (five arms), hexafurcated (six arms)).

A quantity of furcations or arms of the furcated stent may be equal to or less than a quantity of furcations or limbs of the elongated first tube (102) of the delivery catheter. Each furcation or arm of the furcated stent is disposed in a furcation or limb lumen of the elongated first tube (102).

A quantity of furcations or arms of the furcated stent may be equal to or less than a quantity of furcations or divisional branches of the bodily vessel.

The furcated stent (200) may adopt an expanded or compressed configuration. The compressed configuration is for passage within a delivery catheter (110) through a bodily vessel wherein the stent arms (220, 222) and stem (226) have a narrowed transverse cross-sectional profile. The expanded configuration is adopted after deployment; the stent arms and stem have an increased transverse cross-sectional profile. The expanded configuration contacts the inner lumen walls of the bodily vessel. It is understood that in situ, the furcated stent (200) may not be fully expanded; it is generally disposed in a transition state between fully expanded and fully compressed wherein the furcated stent (200) applies a radial force to the vessel wall. Generally, the expanded configuration when discussed herein may be also taken to mean the aforementioned transition state without full expansion.

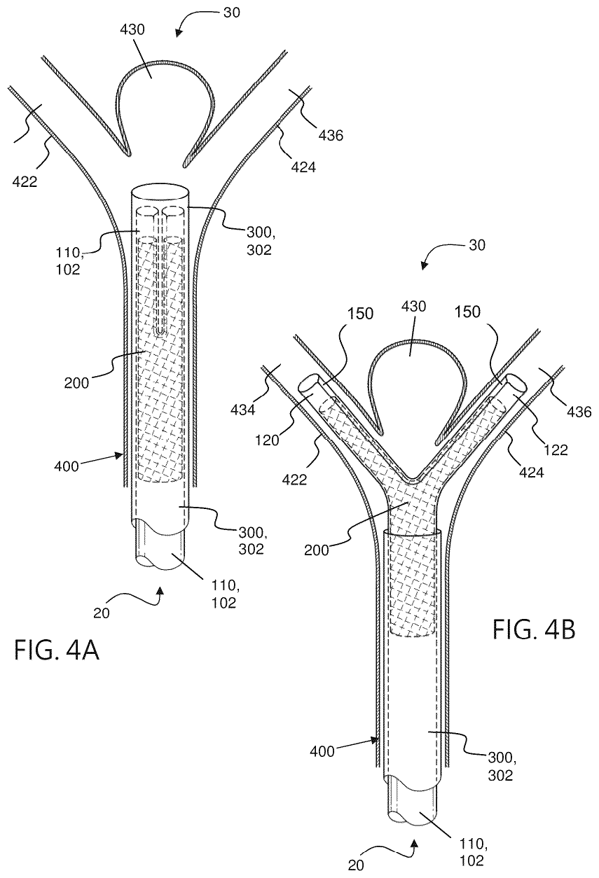

The furcated stent may be self-expanding and may be biased in the expanded configuration; no application of force is required to maintain the expanded configuration. When an external radial force is applied, the furcated stent arms (220, 222) and stem (226) may each be radially compressed, thereby reducing the transverse cross-sectional profile (e.g. diameter) of the stent arms and stem in the compressed configuration. The self-expanding furcated stent may be retained in the compressed configuration within the first lumen (130) of the first tube (102) as described later below. The furcated stent in the compressed configuration is exemplified in FIG. 4A, and in the expanded configuration in FIG. 4D. The self-expanding stent may be formed from a shape memory material such NiTinol, chrome-cobalt alloy, or a biodegradable material.

According to one aspect, the furcated stent may be non-self-expanding (e.g. balloon-expandable). The furcated stent may be initially in a compressed configuration thereby having a reduced transverse cross-sectional profile (e.g. diameter) of the stent arms and stem in the compressed configuration. Application of force is required to transition the compressed furcated stent into the expanded configuration. When a radial force is applied (inner to outer radial direction), for example by a balloon, the furcated stent arms (220, 222) and stem (226) each radially expand, thereby increasing the transverse cross-sectional profile (e.g. diameter) of the stent arms and stem into the expanded configuration. The non-self-expanding furcated stent may be disposed in the compressed configuration within the first lumen (130) of the first tube (102). The non-self-expanding stent may or may not be radially compressed by the stent arms and stem in the compressed configuration. A balloon-expandable furcated stent may be mounted on a balloon for delivery to the site of treatment and to actuate expansion.

The furcated stent (200) may further adopt an open or folded configuration. The folded configuration is for passage within an access catheter (300) through a bodily vessel wherein the stent arms (220, 222) are closer together, typically having an essentially “I”-shaped profile. The open configuration is adopted after deployment within the bodily vessel, the stent arms (220, 222) disposed wider apart (arms spread out). For a bifurcated stent, for example, the stent in the open configuration typically has an essentially “Y”-shaped profile.

The furcated stent (200) may be compliant and biased in the open configuration. Upon the application of force the stent transitions into the folded configuration. With the release of the force, the furcated stent (200) returns to the open configuration. The furcated stent (200) may be retained in the closed (“I”-shaped profile) configuration within the second lumen (330) of the second tube (302) as described later below. The furcated stent (200) is exemplified for a bifurcated stent in the closed configuration in FIG. 4A, and in the open configuration in FIG. 4B.

A furcated stent wall extends along the stem (226) at the proximal end (20) to each of the arms (220, 222) at the distal end (30) defining a stent lumen. The stent lumen (230) is dimensioned in the expanded configuration for the flow of bodily fluid e.g. air, liquid, blood, bodily fluid, secretions, excretions. The stent lumen may be dimensioned in the compressed configuration for the slidable passage of one or more, such as two, guidewires. More in particular, the stent lumen (232) in the stem part (226) may be dimensioned in the compressed configuration for the slidable passage of two guidewires, and the stent lumen (234, 236) of the arms (220, 222) may be dimensioned in the compressed configuration for the slidable passage of one guidewire each.

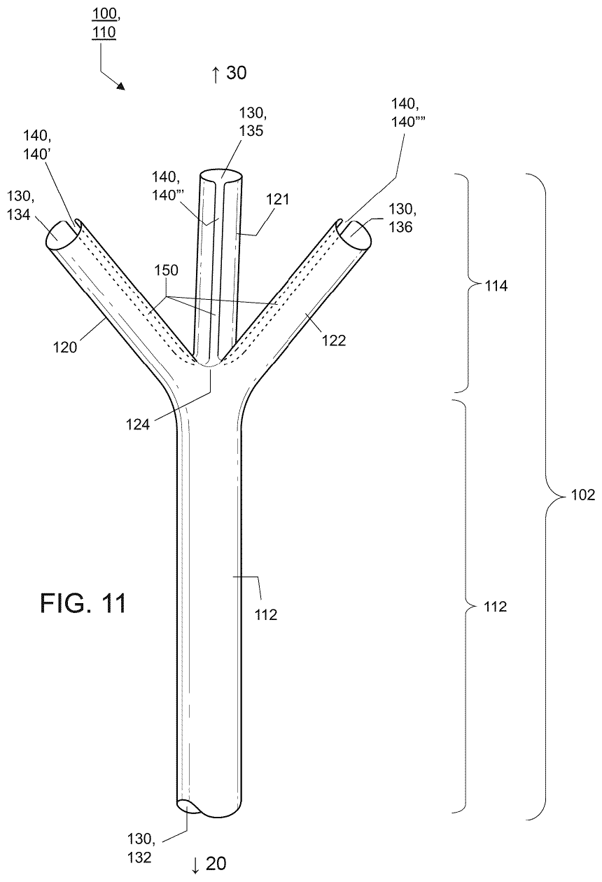

The furcated stent arms may be of equal length in the unfolded condition. Alternatively, the furcated stent arms may be of unequal length in the unfolded condition. Exemplary configurations A to G of a furcated stent that is a bifurcated stent are shown in FIG. 10.

The furcated stent wall may or may not be permeable to blood flow. It may present a variable porosity along the length of the stent allowing to use the furcated stent as a scaffold for the filling of the aneurysm (e.g., as stent-assisted coiling technique) or as a flow diverter stent. The porosity may vary according to its compression or stretching during its deployment. An exemplary configurations of a furcated stent that is a bifurcated stent having an arm (224′) of variable porosity is shown configuration H in in FIG. 10.

The furcated stent (200) may or may not be drug-eluting. According to one aspect, the furcated stent is provided with an active pharmaceutical ingredient having an antithrombotic property, or anticoagulant property, or endothelisation property, or that is a stimulator of cell migration, or a stimulator of cell growth. The active pharmaceutical ingredient may be provided on an inner surface and/or an outer surface of the furcated stent (200).

A different active pharmaceutical ingredient may be provided on an inner surface compared with an outer surface of the furcated stent (200). For instance, an inner surface may be provided with an active pharmaceutical ingredient that promotes patency of the vessel, while an outer surface may be provided with an active pharmaceutical ingredient that promotes thrombosis, coagulation or healing.

According to one aspect, the furcated stent is provided with an active pharmaceutical ingredients at its inner surface having an antithrombotic property, or anticoagulant property, or pro-endothelisation property, or that is a stimulator of cell migration, or a stimulator of cell growth, and at its outer surface having a prothrombotic property, or procoagulant property, or pro-endothelisation property, or that is a stimulator of cell migration, or a stimulator of cell growth.

The furcated stent (200) may be manufactured by braiding wires or by laser-cutting techniques as is known in the art. For aneurysm treatment, the stent allows for blood flow redirecting (i.e., diversion) and therefore promotes thrombus formation inside furcation aneurysms and their occlusion.

Typically, the delivery catheter (110) comprises an elongated first tube (102) having a proximal end (20) and a distal end (30) and furcated part (114) at the distal end (30). The elongated first tube (102) furcates into two or more limbs (also known as tubular lumens) (120, 121, 122, 123) at the distal end. The two or more limbs (120, 121, 122, 123) are mutually connected at a furcation point (124) and to the remainder of the first tube (102) proximal to the furcation point (124). The remainder of the first tube (102) proximal to the furcated part (114) is also known as the main part (112) of the first tube (102). The furcated part (114) comprises at least a first limb (120) and a second limb (122).

The elongated first tube (102) may be bifurcated (one pair of limbs or tubular lumens), or trifurcated (three limbs), or quadfurcated (four limbs or tubular lumens) in the furcated part (114). It is understood the furcated part (114) may contain more than four furcations (e.g. pentafurcated (five limbs or tubular lumens), hexafurcated (six limbs or tubular lumens)).

The first tube (102) is disposed with a first (tubular) lumen (130). The first lumen (130) is in fluid connection with a proximal end (20) of the main part (112) of the first tube (102) and furcates (134, 135, 136, 137) at the distal end (30), corresponding to the furcation of the first tube (102). The furcated part (114) of the first lumen (134, 135, 136, 137) is configured to accommodate the arms (220, 222) of the furcated stent (200). The main part (112) of the first lumen (132), is configured to accommodate the stem (226) of the furcated stent (200), in particular, immediate distal to the furcation point (124). The main part of the first lumen (132) may be further configured for the passage of one or more guidewires, such as multiple guidewires, one for each of the furcated limbs (120, 121, 122, 123). The ends of the delivery catheter (110) may be open for the passage of the one or more guidewires.

The furcated part (114) of the first tube (102) accommodates the arms (220, 222) of the furcated stent (200). The first tube (102) may be adapted to hold the furcated stent (200) (self-expanding or non-self-expanding) in a radially compressed state. Where the furcated stent (200) is self-expanding, the first tube (102) is adapted to maintain the furcated stent (200) in a contracted state. Resistance to expansion of the walls of the first tube, in particular in the furcated part (114), prevents expansion of the furcated stent, maintaining it in contracted state prior to deployment. After the furcated stent (200) is released from the first tube (102) through the slit (140, 140′, 140″, 140″, 140″), the stent expands and occupies the furcated vessel lumen.

Each limb (120, 121, 122, 123) of the furcated part is provided with a slit (140, 140′, 140″). The furcated part (114) contains multiple slits (140, 140′, 140″, 140″, 140″) on multiple limbs (120, 121, 122, 123). Each slit of the multiple slits (140, 140′, 140″, 140″, 140″) is in mutual contact with a least one other slit of the multiple slits. The multiple slits may converge where the respective limbs (120, 121, 122, 123) are connected.

Each slit (140, 140′, 140″, 140″, 140″) may extend from each limb (120, 121, 122, 123) in a proximal (20) direction from a distal (30) terminal end of the limb (120, 121, 122, 123) and the joins where the respective limbs (120, 121, 122, 123) are connected.

Each slit (140′, 140″, 140″, 140″) may contact or cross a central axis of the main part (112) of the first tube (102). The slits on the limbs may or may not be mutually facing. The slit may be aligned straight and parallel with respect to a central axis of the corresponding limb, or inclined with respect to the central axis, or be partially helical. The slit may be continuous. A slit has a path in a proximal to distal (or vice versa) of the limb. The path of a slit be any suitable shape. The path of a slit may be a straight line. The path of a slit may contain one or more straight lines, may contain one or more curves, and/or may contain one or more corners. The path of a may have or contain a wavy shape, may have or contain a zig-zag shape, may have or contain a castellated shape.

Where first tube (102) is, for example, bifurcated, the slit extends from the first limb (120) to the second limb (122). The slit may extend in a proximal (20) direction from a distal (30) terminal end of the first limb (120) and in a proximal (20) direction from a distal terminal end of the second limb (122) and joins where the respective limbs (120, 122) are connected.

Each slit connects the furcated part of first lumen (134, 136) with an exterior of the first tube (102). Each slit (140, 140′, 140″, 140′ “, 140”) may be radial with respect to a central axis of each limb. The furcated part of the first tube (102) releasably retains the furcated stent for deployment after the limbs (120, 121, 122, 123) have been positioned within the furcation of the bodily vessel. The slits (140, 140′, 140″), may be further configured for the passage of a guidewire therethrough.

The slits (140, 140′, 140″, 140′ “, 140”) are together arranged to form a passageway (150) for passage therethrough of the furcated stent. Each slit may extend from each limb in a proximal direction from a distal terminal end of the limb, and connects at its proximal end to at least one other slit to form the passageway. According to one aspect, each and every slit extends through or touches a common slit crossing (152), that is a point or a region of the furcated part. The common slit crossing (152) may contact a central longitudinal axis of the main part (112) of the first tube (102).

Passageway has a narrowed and widened state. In the widened state, the passageway is widened at least in part compared with the narrowed state by the presence of the furcated stent in the passageway. In the widened state, there is an increase in size (area) of a transverse cross-section of a limb at a location compared with a transverse cross-section of the limb at the same location in the narrowed state. The passageway (150) is repeatably widenable. The passageway can repeatably transition between the narrowed and widened state (under application and/or withdrawal of mechanical force). Passageway is biased in the narrowed state. By biased in the narrowed state, it is meant that the narrow state is a stable (native) state of passageway attained without application of mechanical force. Application of mechanical force causes a deformation of the passageway; removal of the force causes the passageway to return to the narrowed state. The ability of the passage to revert to the narrowed state might be known as elasticity.

The furcated part (114) is configured such that the bias of the passageway in the narrowed state is overcome by passage of the furcated stent through the passageway (under application of mechanical force). In particular, the passageway is configured to transition between the narrowed state and widened state by application and/or withdrawal of force between the passageway and the furcated stent.

In an aspect, the narrowed state each limb (120, 121, 122, 123) is biased in an essentially tubular shape for retention of the furcated stent. More in particular, in the narrowed state each limb (120, 121, 122, 123) is biased in an essentially tubular shape for retention of a stent arm (220, 222) within the limb (120, 121, 122, 123) of the furcated part (114). The narrowed state of each limb may extend circumferentially around a majority of a circumference of a respective arm of the arms of the furcated stent.

In a widened state, the bias of the passageway (150) in the narrowed state is overcome by passage of the furcated stent through the passageway (under application of mechanical force). In the widened state, the passageway is opened under force from the furcated stent. The widened state of each limb may extend circumferentially around less than in the narrowed state. When the application of force is removed, the passageway reverts to the stable narrowed state.

The slit (140, 140′, 140″, 140″, 140″) may be open (apart) or closed in the biased narrowed state of the passageway (150).

In the slit closed state where the passageway is in the narrowed state, the slit edges may touch (e.g. FIG. 1B′-1, 142′ and FIG. 1B-1, 142″) or overlap (e.g. FIG. 1B′-2, 144′ and FIG. 1B-2, 144″).

Where the slit is open (apart) and the passageway is in the narrowed state, the edges of the slit do not touch. The open (apart) slit (140, 140′, 140″, 140″, 140″) may occupy a between 1-30% of the circumference of the limb (120, 121, 122, 123) in the biased narrowed state of the passageway. Where the open (apart) slit (140, 140′, 140″, 140″, 140″) is present, the circumference of the limb (120, 121, 122, 123) wall may occupy a majority of the circumference of the limb in the biased narrowed state of the passageway. The width of the open (apart) slit may be adapted allow ease of passage by the furcated stent, while providing sufficient resistance to expansion of the walls to retain a self-expanding furcated stent in a closed state, or to apply a compressive force to a non-self-expanding furcated stent.

The slit edges, whether in the open or closed state, may detached from each other. The slit has edges may be devoid of a material connection (i.e. breakable seal) there between. The slit edges may be devoid of a material connection there between that restricts movement (e.g. opening or closing) of the slit edges relative to each other.

The furcated part (114) of the first tube (102) may be formed from a compliant material biased to as to form the passageway in the narrowed state. The compliant material may be an elastic material. The furcated part (114) of the first tube (102) may be compliant and biased in the essentially tubular configuration to allow the repeatable passage (advancement and withdrawal) through the passageway of the furcated stent therethrough. For instance, the furcated stent may be deployed by withdrawal of the delivery catheter relative to the furcated stent. For instance, the furcated stent may be re-sheathed by advancement of the delivery catheter relative to the furcated stent. The ability to unsheath and re-sheath the furcated stent allows the furcated stent to be repositioned after an initial deployment by the delivery catheter at a non-optimal location.

The furcated part (114) of the first tube (102) may be configured to apply radial force to the furcated stent disposed in the lumen of furcated part (114) of the first tube (102), in particular when the passageway is in the narrowed state. Where the furcated stent is self-expanding, the radial force maintains the furcated stent in the compressed state and resists expansion. Where the furcated stent is non-self-expanding, the radial force compresses the furcated stent.

One or more slits slit may be disposed with one or more breakable seals, configured to prevent passive passage of the furcated stent therethrough and/or to prevent expansion of a self-expanding furcated stent. The breakable seal may be broken as the delivery catheter (110) is withdrawn from the furcated stent (200) or as the furcated stent (200) is advanced using the pusher (500). The breakable seal may be formed by a region of reduced wall thickness of the first tube (102); the region of reduced wall thickness may span part of all of the slit. The breakable seal may be formed by a region where the slit is interrupted i.e. the edges of the slit are bridged by a non-cut part.

The slit edges, whether in the open or closed state, may detached from each other. The slit has edges may be devoid of a material or mechanical connection (i.e. breakable seal) there between. The slit edges may be devoid of a material or mechanical connection there between that restricts movement (e.g. opening or closing) of the slit edges relative to each other.

The slit may be formed by laser cutting, water cutting or milling of the wall of the first tube in the furcated part (114) which also allows formation of the breakable seal by, for instance, partial cutting to a certain depth and/or by providing the slit with interruptions (bridges). The slit may be formed by molding or extrusion.

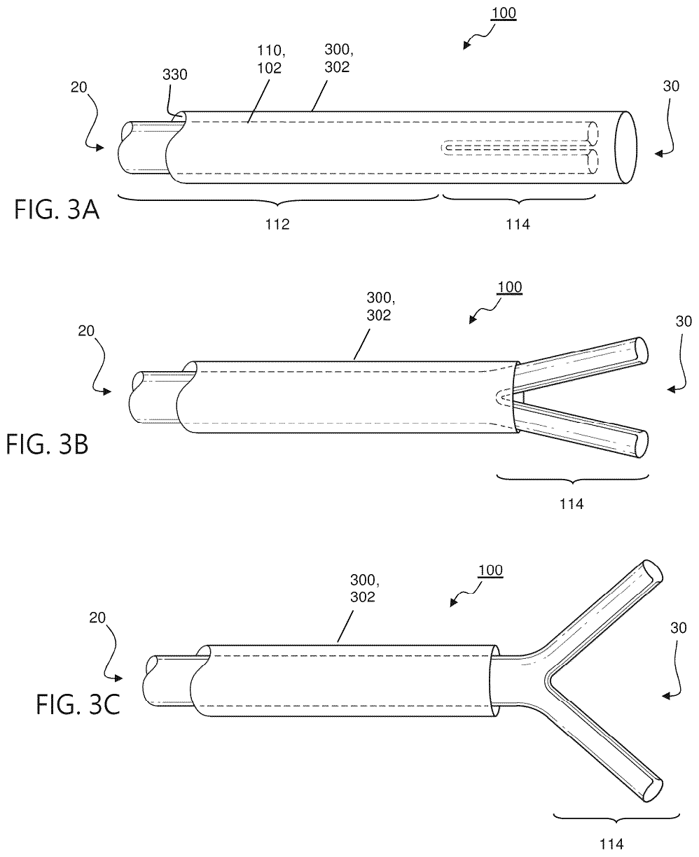

The furcated part (114) of the first tube (102) is compliant and biased in an open (limbs spread out) configuration. Upon the application of an external force, the furcated part (114) may transition into a folded configuration in which the respective distal ends of the limbs (120, 121, 122, 123) brought closer together. With the release of the force, the furcated part (114) returns to the open (limbs spread out) configuration. The foldable property allows slidable passage through a constricting lumen (330) of an access catheter (300). The furcated part (114) of the first tube (102) is exemplified for a bifurcated part (114) in the closed configuration in FIG. 3A, and in the open configuration in FIG. 3C.

Controlled and gradual opening and/or folding of the furcated part (114) of the first tube may be actuated by a withdrawal of or advancement through a constricting second lumen (330) of an access catheter (300) described in more detail later below, namely sheathing or unsheathing of the furcated part (114) of the first tube. The access catheter (300) may be withdrawn while the first tube (102) is maintained in an essentially fixed relation to the site of treatment. Advantageously, the furcated part (114) of the first tube may be resheathed and repositioned where it has not been satisfactorily deployed or placed.

The first tube (102) may be sized for slidable passage through, for example, the working channel of an endoscope or a second lumen of the second tube (described below). As a general guidance, for vascular applications, the maximum outer diameter of the main part (112) of the first tube (102) may be equal to or no greater than 0.1 F to 0.3 F (0.05 mm to 0.10 mm).

As a general guidance, the maximum diameter of the first lumen (132) of the main part (112) of the first tube (102) may be equal to or no greater than 0.1 F to 0.2 F (0.04 mm to 0.07 mm).

As a general guidance, the length of the main part (112) of the first tube (102) may be 120 to 160 cm, depending on the application. The respective diameters and length may be adapted according to the location with respect to the point of entry, dimensions of the vessel to be treated e.g. artery size, size of aneurism neck, and the anatomy.

The maximum outer diameter of the main part (112) of the first tube (102) may be greater than the maximum outer diameter of the 120, 121, 122, 123 limbs (120, 121, 122, 123). The maximum outer diameter of the one limb of the plurality of limbs may be the same or different from the maximum outer diameter of a remainder of the limbs. The respective diameters may be adapted according to the dimensions of the vessel to be treated e.g. artery size, size of aneurism neck.

As a general guidance, for vascular applications (e.g. intracranial), the maximum outer diameter of each of the limbs (120, 121, 122, 123) of the first tube (102) may be equal to or no greater than 0.1 F to 0.2 F (0.04 mm to 0.07 mm).

As a general guidance, the maximum diameter of the first lumen (136, 134) of the first (120) or second (122) limb of the first tube (102) may be equal to or no greater than 0.09 F to 0.18 F (0.03 mm to 0.06 mm).

The length of the one limb of the plurality of limbs may be the same or different from the length of the remainder of the limbs. The respective lengths may be adapted according to the dimensions of the vessel to be treated e.g. artery size, size of aneurism neck.

The lengths of the limbs (120, 121, 122, 123) may be longer than the respective lengths of the arms (220 222).

The first tube (102) may be formed using an extrusion process or non-extrusion process. A first tube may be formed from a biocompatible material which provides the requisite flexibility, pushability and strength. It may also exhibit low or no radial expansion. Suitable biocompatible materials include, but are not limited to a polymer such as polypropylene, polyethylene, polyurethanes, polyamide, polyimide poly (ethylene terephthalate) (PET) or polyesters and copolymers thereof, metal (stainless steel, NiTinol) of a combination of metal and polymer. The first tube may formed from a polymeric material that is polyamide, polyimide, stainless steel or NiTinolor a combination or blend of these. The first tube may be formed from a polymeric material (e.g. polyimide) strengthened with braided or coiled metal (stainless steel or NiTinol) disposed within the polyimide wall. For a first tube formed by extrusion, the first tube may be formed from polyamide. For a first tube formed by non-extrusion, the first tube may be formed from polyimide. The exterior may be coated to reduce friction during insertion or withdrawal. Example of a suitable friction-reducing coating includes Teflon.

The main part (112) of the first tube (102) may be provided with a coiled spring disposed at least partially along a length to increase stiffness and pushability, while maintaining flexibility. The coiled spring may be disposed adjacent to an inner wall of the first lumen. A rubber layer may be provided to protect the coiled spring. The rubber layer may be provided with a friction-reducing coating e.g. a hydrophilic polymer for ease of passage of a guidewire through the first lumen. It is an aspect that the furcated part of the first tube is devoid of the coiled spring; stiffness may be provided by the presence of the furcated stent.

The delivery catheter (110) may further be provided with one or more expandable balloons. The expandable balloon may be used for the treatment of an occlusion and/or for opening of the furcated stent that is a balloon-expandable non-self-expanding furcated stent. The expandable balloon may be advanced towards the site of treatment via the first lumen (230) of the first tube (102). The expandable balloon and associated catheter may be slidable relative to the delivery catheter (110). In a particular example, an expandable balloon may be employed in order to perform intrastent angioplasty in case of non-full deployment of the furcated stent owing to anatomic complexity. The furcated stent may be mounted on balloons-one for each stent arm—that allows the deployment of a balloon-expandable stent.

The first tube (102) may comprise one or more additional lumens, for instance, for deployment of an expandable balloon, which may be used to perform in-stent angioplasty.

The treatment of arterial occlusion may be performed by 1) deploying the furcated stent through the clot (or thrombus), 2) pausing for a period in order to allow the clot to penetrate the struts of the furcated stent and 3) by pulling back (retrieving) the furcated stent with the clot that penetrated into its struts.

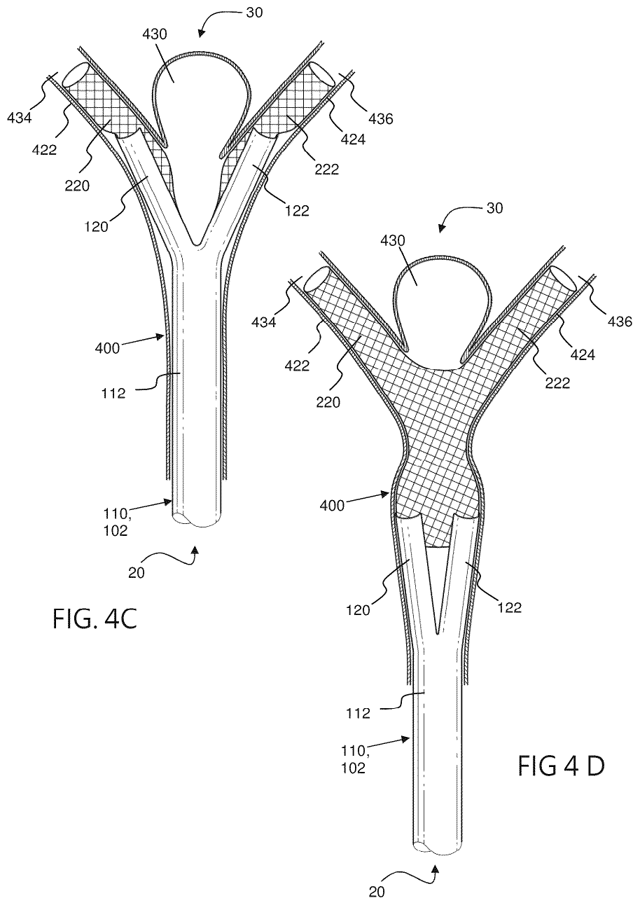

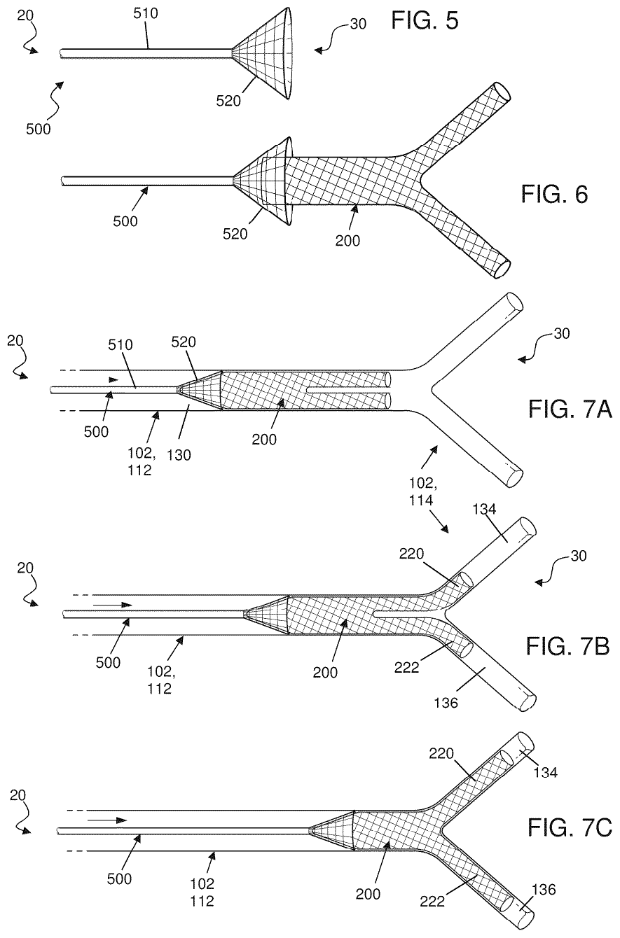

The delivery catheter (110) may further be provided with a flexible and slidable pusher (500) to assist with dispensing the furcated stent through the passageway, as shown, for instance, in FIGS. 5 and 6. The pusher (500) comprises an elongated flexible rod (510) having a proximal (20) and distal (30) end, configured for passage through the first lumen (130) of the first tube (102). The pusher (500) may be slidable relative to the delivery catheter.

The pusher rod (510) may be formed from a biocompatible material which provides the requisite flexibility, pushability and strength. Suitable biocompatible materials include, but are not limited to a polymer such as polypropylene, polyethylene, polyurethanes, polyamide, polyimide poly (ethylene terephthalate) (PET) or polyesters and copolymers thereof. The rod exterior may be coated to reduce friction during passage through the first lumen (230) of the first tube (102). Examples of a suitable friction-reducing coating includes Teflon. The pusher rod may be formed from a hollow tube or may be at least partially, such as fully solid.

According to one aspect, the distal end (30) of the pusher rod (500) may be permanently attached to the furcated stent (200) at its proximal end (20); for instance, when the furcated stent is used to treat a vascular occlusion by removal of a part of the occlusive material, there is no requirement for the furcated stent (200) to be released.

According to one aspect, the distal end (30) of the pusher rod (500) is provided with a capture element (520) for releasable attachment to the furcated stent (200) at its proximal end (20). The capture element (520) may be provided in fixed slidable and preferably rotational relation with the distal end of the pusher rod. The capture element (520) may be radially self-expanding e.g. a radially self-expanding claw or net that may adopt an open or folded configuration. The folded configuration is for passage within the first lumen (130) of the first tube (102) wherein the peripheral edges of the capture element (520) are closer together, typically having an essentially “I”-shaped profile. The capture element (520) in the folded configuration is able to grip the proximal end (20) of the furcated stent (200); once gripped, translations and rotations of the pusher rod are transferred to the furcated stent (200). The open configuration is adopted after deployment of the furcated stent (200) the peripheral edges being disposed wider apart, typically having an essentially conical or dome-shaped form. The capture element (520) in the open configuration releases the grip of the proximal end (20) of the furcated stent (200).

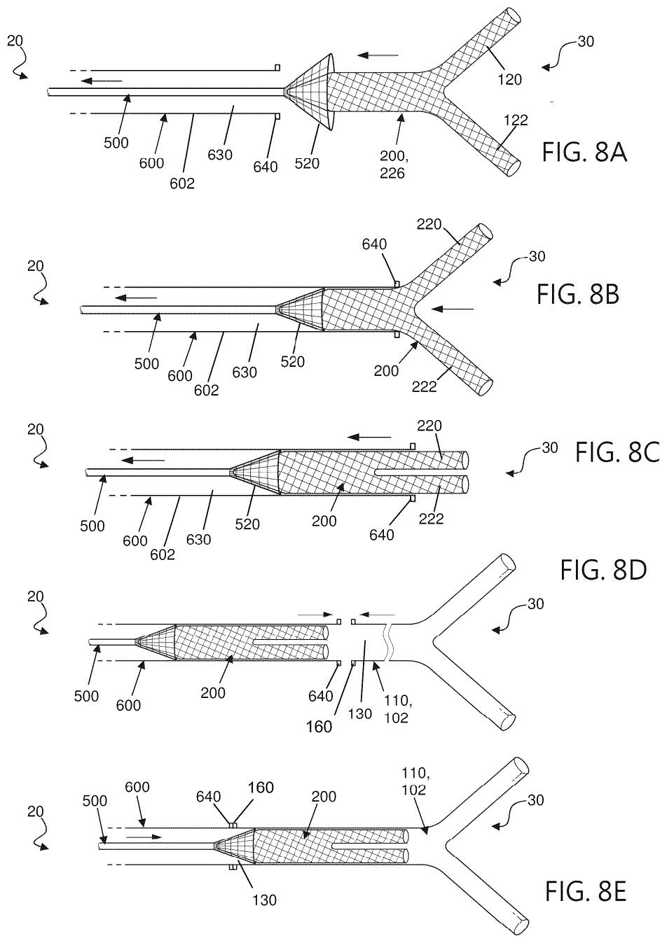

The capture element (520) may be compliant and biased in the open (conical or dome-shaped-shaped) configuration. Upon the application of radial force the capture element (520) transitions into the folded configuration. The capture element (520) may be retained in the closed (“I”-shaped profile) configuration within the first lumen (130) of the first tube (102). With the release of the radial force, the capture element (520) returns to the open configuration. The capture element (520) may adopt the open configuration after it has been advanced through the passageway of the furcated part (114) of the first tube. The capture element (520) in the closed configuration is exemplified in FIGS. 7A to C, and in the open configuration in FIGS. 5 and 6.

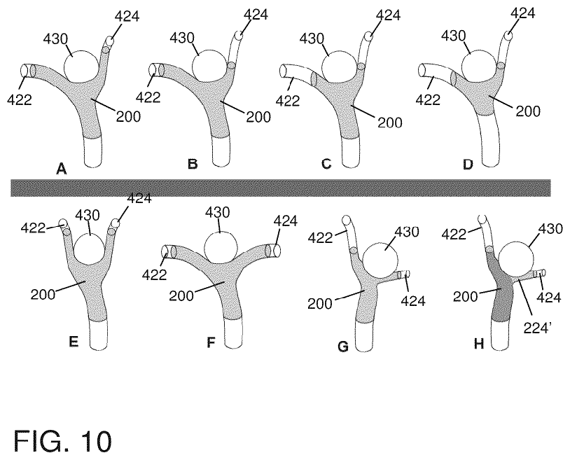

Advancement of the pusher (500) in a distal (30) direction while the position of the delivery catheter (110) is maintained, or withdrawal of the delivery catheter (110) while the position of the pusher (500) is maintained allows the furcated stent (200) to be deployed i.e. ejected from the slit. FIGS. 9A and 9B depict a sequence where the pusher (500) is maintained at a constant position while the delivery catheter (110) is withdrawn proximally, thereby deploying the furcated stent (200).

It is noted that the pusher (500) may be utilized for loading the furcated stent (200) into the limbs (120, 121, 122, 123) of the delivery catheter (110). A loading sequence is depicted, for instance, FIGS. 7A to 7C. In the folded configuration, the furcated stent (200) may be introduced into the proximal end (20) of the first lumen (130) of the main part (112) of the first tube (102), where, being gripped by the capture element (520) in the closed configuration, it is advanced in a distal (30) direction along first lumen (130) of the main part (112) as shown in FIG. 7A. Where it reaches the furcated part (114) of the first tube (102), the furcated stent (200) arms (220, 222) unfold into and occupy the lumen (134, 136) in the furcated part (114) of the first tube (102) (FIGS. 7B and 7C). It is appreciated that loading of the furcated stent (200) into the delivery catheter (110) may be performed over a pair of guidewires.

The delivery system (100) for the furcated stent may further comprise an access catheter (300) for delivering the delivery catheter (110) to the site of treatment, as shown, for instance in FIG. 3A to 3C.

The access catheter (300) comprises an elongated second tube (302) having a proximal end (20) and a distal end (30) provided with a second lumen (330) adapted to slidably accommodate the delivery catheter (110) or first tube (102). The second tube (302) is configured to slidably accommodate the furcated part (114) of the first tube (102) in a folded configuration.

The proximal (20) and distal (30) terminal ends of the second tube (302) are open. The second tube (302) may be cylindrical, having a generally uniform outer shape in the proximal region. It will be appreciated that an open proximal end may be configured for connection to one or more hubs. One or more hubs such as a Y-type connector, optionally with Luer fittings may be fitted to the proximal terminal end of the access catheter or second tube to facilitate passage of the first tube or delivery catheter, guidewire, equipment to provide torque/longitudinal force via the first tube.

As would be understood by those of skill in the art, the second tube (302) may be sized for slidable passage through, for example, the working channel of an endoscope or through a body lumen, in particular vasculature (through an introducer). As a general guidance, for vascular applications (e.g. intracranial), the maximum outer diameter of the second tube (302) towards the distal (in situ) end may be equal to or no greater than 4 F to 6 F (1.33 mm to 2 mm).

As a general guidance, the maximum diameter of the second lumen (330) the distal (in situ) end may be equal to or no greater than 3.9 F to 5.9 F (1.30 mm to 1.97 mm).

As a general guidance, the length of the second tube (302) may be 110 to 150 cm, depending on the application.

The second tube (302) may be formed using an extrusion process or non-extrusion process. A second tube (302) may be formed from a biocompatible material which provides the requisite flexibility, pushability and strength. Suitable biocompatible materials include, but are not limited to a polymer such as polypropylene, polyethylene, polyurethanes, polyamide, polyimide poly (ethylene terephthalate) (PET) or polyesters and copolymers thereof, metal (stainless steel, NiTinol) of a combination of metal and polymer. The second tube may be formed from a polymeric material that is polyamide, polyimide, stainless steel or NiTinolor a combination or blend of these. The second tube (302) may be formed from a polymeric material (e.g. polyimide) strengthened with braided or coiled metal (stainless steel or NiTinol) disposed within the polyimide wall. For a second tube (302) formed by extrusion, may be formed from polyamide. For a second tube (302) formed by non-extrusion, may be formed from polyimide. The exterior may be coated to reduce friction during insertion or withdrawal. Example of a suitable friction-reducing coating includes Teflon.

The second tube (302) may be provided with a coiled spring disposed at least partially along a length to increase stiffness and pushability, while marinating flexibility. The coiled spring may be disposed adjacent to an inner wall of the second lumen. A rubber layer may be provided to protect the coiled spring. The rubber layer may be provided with a friction-reducing coating e.g. a hydrophilic polymer for ease of passage of the first tube through the second lumen.

The second tube (302) may comprise one or more additional lumens, for instance, for deployment of an expandable balloon for the treatment of an occlusion.

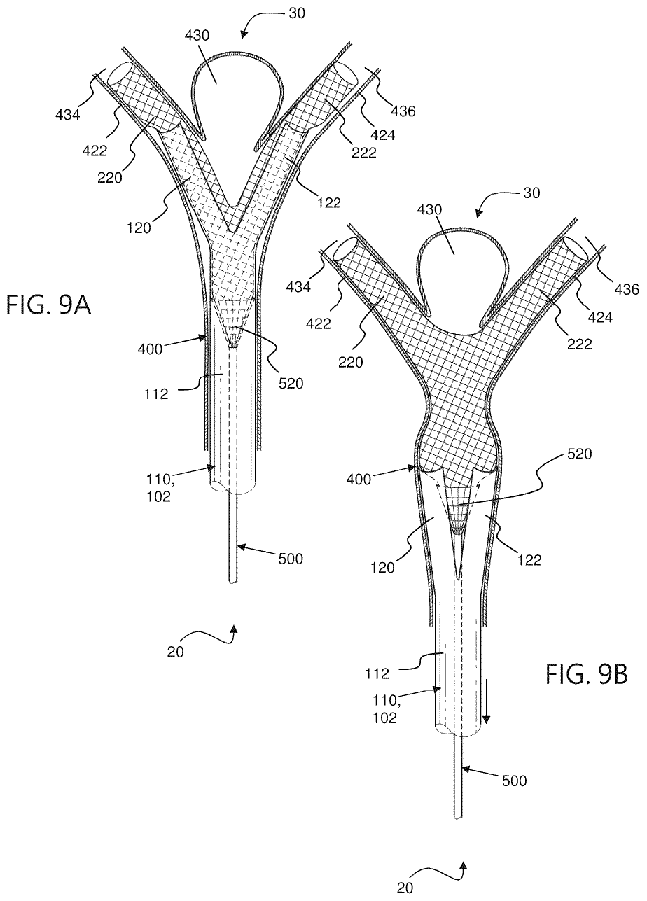

The delivery system (100) may further comprise a loader (600) configured for loading of the furcated stent (200) into the delivery catheter (110), as shown, for instance in FIGS. 8A to 8E.

The loader (600) comprises an elongated third tube (602) having a proximal end (20) and a distal end (30) provided with a third lumen (630) adapted to slidably accommodate the furcated stent (200) in the folded configuration.

The distal end (30) of the third tube (602) is configured to couple with the proximal terminal end of the delivery catheter (110) such that the first (130) and third (630) lumens are connected to form a continuous passage for advancement of the furcated stent (200) in the folded state from the loader (600) to the delivery catheter (110). The proximal (20) and distal (30) terminal ends of the third tube (602) are open. The distal end (30) of the third tube (602) may be provided with a coupling (640) (e.g. a Luer filling, a push connector, a narrowed region of the third tube (602)) configured to connect with a complementary coupling (160) provided on the proximal end of the first tube (102) such as a hub.

The third tube (602) may be cylindrical, having a generally uniform outer shape in the distal region.

The proximal end (stem part (226)) of the furcated stent (200) is inserted into the distal end of the third (630) lumen, and the furcated stent (200) withdrawn proximally (20) so as to fold the arms (220, 222) as the third tube (602) covers them (FIGS. 8A to 8C). Subsequently, the distal end (30) of the third tube (602) is coupled to the proximal end of the first tube (102) such that the respective lumens (630, 130) form a continuous passage (FIG. 8D). Subsequently, the furcated stent (200) is advanced distally (30) such that it enters the first lumen (130) of the first tube (102) (FIG. 8 E). The loader (600) thus facilitates loading of the furcated stent (200) from the proximal end of the delivery catheter (110) wherein the arms (220, 222) are oriented in a distal (30) direction.

It is appreciated that the pusher (500) or other type of rod may be used to advance and withdrawal of the furcated stent (200) relative to the loader (600).

The delivery system (100) may be provided with at least one guidewire, such as two, three, or four guidewires or no more than 6. The quantity of guidewires may be equal or less than to the quantity of furcations in the bodily vessel, or in the stent, or in the first tube. The guidewire may have a shapeable distal end for intraluminal navigation.

There are two main types of catheters in common use-rapid exchange (monorail) and over the wire (OTW). Over the wire catheters employ a long guidewire lumen from the proximal end to the distal end of the catheter. Rapid exchange catheters employ a distal guidewire lumen, having a side port for exits of the guidewire towards the distal end. The fact that the guidewire is received only within a distal portion allows the catheter to be readily exchanged without the need for guidewire extenders or for an excessively long guidewire. The present delivery system can be readily adapted for guidewire deployment using either mode. The figures demonstrate an OTW mode, however, it is readily within the competence of the skilled person to adapt it for the rapid exchange mode of operation.

The delivery system (100) may comprise the delivery catheter (110) and one or more of the following elements: the furcated stent (200), the access catheter (300), or one or more guidewires.

The delivery system (100) may be provided as a kit comprising the delivery catheter (110) and one or more of the following elements: the furcated stent (200), the access catheter (300), or one or more guidewires.

The delivery system (100) may comprise the delivery catheter (110) and one or more of the following elements: the furcated stent (200), the access catheter (300), the pusher (500), the loader (600), or one or more guidewires.

The delivery system (100) may be provided as a kit comprising the delivery catheter (110) and one or more of the following elements:—the furcated stent (200), the access catheter (300), the pusher (500), the loader (600), or one or more guidewires.

It is appreciated that the delivery system (100) or kit may be supplied wherein the delivery catheter (110) and one or more of the elements are not co-assembled.

A quantity of furcations, catheter first tube limbs, stent arms (where stent is present) and guidewires (where present) may be no more than the number of division branches of the bodily vessel. A quantity of division branches of the bodily vessel may be no more than 6.

A further aspect described herein relates to a method for delivery of a furcated stent to a site of treatment using the delivery system (100) described herein comprising: advancing intravascularly the access catheter (300) disposed with the delivery catheter (110) loaded with the furcated stent (200) to the site of treatment through the access catheter (300), opening gradually the delivery catheter (110) by withdrawal of the access catheter (300), and deploying the furcated stent (200) through the passageway (150) by withdrawal of the delivery catheter (110).

The delivery system (100) may be advanced intravascularly along the one or more guidewires. Typically, there is one guidewire per vessel branch. Guidewire placement precedes advancement of the delivery system (100).

Further aspect described herein relates to a method for treatment of an arterial aneurism located at a vessel furcation using the delivery system (100) described herein comprising:
advancing intravascularly the delivery catheter (110) loaded with the furcated stent (200) to the site of the aneurism through the access catheter (300),
opening gradually the delivery catheter (110) by withdrawal of the access catheter (300) such that each limb (120, 121, 122, 123) of the delivery catheter (110) is positioned within a branch of the vessel furcation, and
deploying the furcated stent (200) through the slit by withdrawal of the delivery catheter (110), such that each arm (220, 222) of the furcated stent (200) is positioned within a branch of the vessel furcation.

After deployment, the furcated stent (200) is detached from the delivery system (100), for instance, from the pusher (500), and furcated stent (200) remains in situ.

Further aspect described herein relates to a method for treatment of an arterial occlusion located at a vessel furcation using the delivery system (100) described herein comprising:
advancing intravascularly the delivery catheter (110) loaded with the furcated stent (200) to the site of the occlusion through the access catheter (300),
opening gradually the delivery catheter (100) by withdrawal of the access catheter (300) such that each limb (120, 121, 122, 123) of the delivery catheter (110) is positioned within a branch of the vessel furcation,
deploying the furcated stent (200) through the passageway (150) by withdrawal of the delivery catheter (110), such that each arm (220, 222) of the furcated stent (200) is positioned within a branch of the vessel furcation and one or both arms (220, 222) are positioned within occlusive material (e.g. clot, thrombus, emboli),
withdrawing furcated stent (200) together with at least part of the occlusive material.

Withdrawal of the furcated stent (200) may be effected by a pusher rod attached to the furcated stent (200).

The arterial aneurism or arterial occlusion may or may not be intracranial.

Further aspect described herein relates to a method for loading furcated stent (200) into the delivery catheter (110) comprising the steps:
inserting the proximal end (20) of the furcated stent (200) the distal end of the distal end (30) of the third (630) lumen of the loader (600),
withdrawing the furcated stent (200) proximally (20) so as to fold the arms (220, 222) as the third tube (602) covers them,
coupling the distal end (30) of the third tube (602) to the proximal end of the first tube (102) delivery catheter (110) of the such that the respective lumens (630, 130) form a continuous passage.
advancing the furcated stent (200) distally (30) such that it enters the first lumen (130) of the first tube (102).

By use of the method, the furcated stent (200) is loaded the proximal end of the delivery catheter (110) and the arms (220, 222) are oriented in a distal (30) direction for subsequent deployment. The method may employ a pusher (500) to advance or withdraw the furcated stent (200) relative to the loader (600).

FIG. 1 depicts a delivery catheter (110) in an open configuration having a proximal (20) and distal end (30) comprising a first tube (102) disposed with a first lumen (130, 132, 134, 136). The first tube (102) is furcated, namely bifurcated at the distal end (30) at a furcation point (124), forming a proximal main part (112) disposed with the first lumen (132) and furcated part (114) where the first lumen (134, 136) advances into each of the limbs (120, 122) of the furcated part (114). The furcated part (114) is disposed with a passageway (150) for passage of the furcated stent (200), the passageway (150) formed from two longitudinal slits (140′, 140″), one slit (140′, 140″) disposed in each limb (120, 122).

FIG. 1B and FIG. 1B′ illustrate a transverse cross section through the first limb (120) and second limb (122) respectively, together with the respective slits (140′, 140″) and the respective parts of the first lumen (134, 136). FIG. 1A illustrates a transverse cross section through the main part (112) of the first tube (102).

FIG. 2 depicts a furcated stent (200) that is a bifurcated stent in an open (Y) configuration having a proximal (20) and distal (30) end and comprising a stem part (226) at the proximal end (20) furcating at a furcation point (224) into a pair of arms (220, 222) at the distal end (30). A stent lumen (230) extends from the proximal end (232) of the stem part (226) to the distal end of each arm (234, 236).

FIGS. 3A to 3C each depict an access catheter (300) having a proximal (20) and distal (30) end comprising a second tube (302) disposed with a second lumen (330), wherein the second lumen (330) slidably accommodates the first tube (102). In FIG. 3A, the furcated part (114) of the first tube (102) is fully withdrawn within the second lumen (330) causing the furcated part (114) to adopt the folded configuration. In FIG. 3 B, the furcated part (114) of the first tube (102) is partially withdrawn within the second lumen (330) causing the furcated part (114) to adopt an open configuration. In FIG. 3C, the furcated part (114) of the first tube (102) is fully unsheathed from the second lumen (330) allowing the furcated part (114) to adopt a fully open configuration.

FIGS. 4A to 4D show a delivery and deployment sequence into a portion of an artery (400) having proximal (20) and (30) distal end that is a furcated, namely bifurcated at the distal end (30) into two branches (422, 424) which lead off from a main part (426) of the artery. An arterial lumen (430) has a corresponding lumen main part (432) and lumen branches (434, 436). An abnormality that is aneurism sac (430) has formed where the branches (422, 424) furcate (e.g. bifurcate). In FIG. 4A, the delivery system (100) has been advanced along the main part (426) of the artery and proximal to the furcated part of the artery (400). The delivery system (100) comprises a furcated stent (200) loaded into the furcated part of the delivery catheter (110), and an access catheter (300) into which the delivery catheter (110) is withdrawn to maintain the limbs in a folded configuration. Typically, the delivery system (100) is advanced along two guidewires (not shown), one guidewire for each arm of the furcated stent and threaded through each of the arterial branches. In FIG. 4B, the access catheter (300) has been withdrawn relative to the delivery catheter (110), thereby unsheathing the delivery catheter (110) and opening the limbs (120, 122), each of which has been positioned into a branch (422, 424) lumen (434, 436) of the furcated artery (400). In practice, the unsheathing is done gradually and in tandem with positioning. The delivery catheter (110) may be repeatedly re-sheathed and un-sheathed for optimum placement. In FIG. 4C, the delivery catheter (110) has been partially withdrawn relative to the furcated stent (200), thereby partly dispensing the furcated stent (200) through the passageway (150).

Each arm (220, 222) of the furcated stent (200) is disposed into a branch (422, 424) lumen (434, 436) of the furcated artery (400). Guidewires where used may be immobilised during dispensing. In FIG. 4 D, the delivery catheter (110) has been further withdrawn relative to the furcated stent (200). Each arm (220, 222) of the furcated stent (200) is fully deployed into an arterial branch (422, 424) lumen (434, 436), and the stem (226) is partially deployed. The stent mesh covers the wide neck of the aneurism sac (430).

FIG. 5 depicts a pusher (500) having a proximal (20) and distal (30) end comprising an elongated flexible rod (510) disposed at the distal end (30) with a capture element (520) in an open configuration for releasable attachment to the furcated stent (200) at its proximal end (20).

FIG. 6 depicts the pusher (500) of FIG. 5 where the capture element (520) in an open configuration is in abutting relation to the furcated stent (200) that is a bifurcated stent, and prior to capture.

FIGS. 7A to 7C depict a loading sequence for loading the furcated stent (200) that is a bifurcated stent into the limbs (120, 122) of the delivery catheter (110). In FIG. 7A, the furcated stent (200) in a folded configuration is gripped by the capture element (520) in the closed configuration and advanced in a distal (30) direction along first lumen (130) of the main part (112) of the first tube (102) by pushing the elongated flexible rod (510) in a distal direction. In FIG. 7B, the furcated stent (200) arms (220, 222) start to unfold into and occupy the lumen (134, 136) in the furcated part (114) of the first tube (102). In FIG. 7C, the furcated stent (200) is fully loaded into the furcated part (114) of the first tube (102).

FIGS. 8A to 8E depict a loading sequence for loading the furcated stent (200) that is a bifurcated stent into the delivery catheter (110) such that the arms (220, 222) are aligned in a distal direction. The proximal end (stem part (226)) of the furcated stent (200) is inserted into a capture element (520) of the pusher (500) in the open configuration (FIG. 8A). The pusher (500) is withdrawn proximally (20) into the distal end of the third (630) lumen so as to fold the capture element (520) and grip the stem part (226) of the furcated stent (200) (FIG. 8B). The pusher (500) is further withdrawn proximally (20) so as to fold the furcated stent (200) arms (220, 222) as the third tube (602) covers them (FIGS. 8A to C). After the furcated stent (200) arms (220, 222) have been closed, the distal end (30) of the third tube (602) is coupled via a coupling (640) to the proximal end of the first tube (102) disposed with a complementary coupling (160) such that the respective lumens (630, 130) form a continuous passage (FIG. 8D). Subsequently, the furcated stent (200) is advanced distally (30) by pushing the pusher (500) forward such that furcated stent (200) enters the first lumen (130) of the first tube (102) (FIG. 8E).

FIGS. 9A and B show a deployment sequence into a portion of an artery (400) akin to the sequence shown in FIG. 4, wherein the furcated stent (200) that is a bifurcated stent is maintained or advance relative to the delivery catheter (110) using the pusher (500). In FIG. 9A, the limbs (120, 122) of the delivery catheter (110) have been positioned into a branch (422, 424) lumen (434, 436) of the furcated artery (400). The delivery catheter (110) has been partially withdrawn relative to the furcated stent (200) which is maintained in position maintaining the pusher rod (500) fixed relative to the site of treatment, thereby partly dispensing the furcated stent (200) through the passageway. In FIG. 9B, the delivery catheter (110) has been further withdrawn relative to the pusher rod (500) and furcated stent (200). Each arm (220, 222) of the furcated stent (200) is fully deployed into an arterial branch (422, 424) lumen (434, 436), and the stem (226) is partially deployed. The stent mesh covers the wide neck of the aneurism sac (430).

FIG. 10, show different configurations A to H of a furcated stent (200) that is a bifurcated stent according to the dimension of the arterial branches (422, 424). Compared with configuration A, in configurations B and C, the length of the furcated stent arms are different; in configuration D, the length of the furcated stent stem is different; in configuration E, the branch angle is different as is the diameter of the left branch of the furcated stent; in configuration F, the branch angle is different as is the diameter of the right branch of the furcated stent; in configuration G, the branch angle is different, as are the diameters and length of the branches. In configuration H, similar to configuration G, the porosity of the right stent branch varies at region 224′.

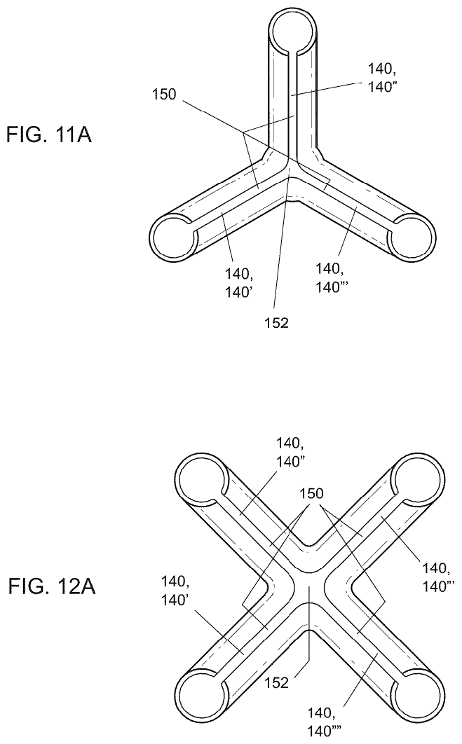

FIG. 11 depicts a delivery catheter (110) in an open configuration having a proximal (20) and distal end (30) comprising a first tube (102) disposed with a first lumen (130, 134, 135, 136). The first tube (102) is furcated, namely trifurcated at the distal end (30) at a furcation point (124), forming a proximal main part (112) disposed with the first lumen (132) and furcated part (114) where the first lumen (130, 134, 135, 136) advances into each of the limbs (120, 121, 122) of the furcated part (114). The furcated part (114) is disposed with a passageway (150) formed from a joining of longitudinal slits (140′, 140″, 140″) in each limb (120, 121, 122) for passage of a trifurcated stent.

FIG. 11A illustrates a view of the delivery catheter of FIG. 11 in a distal (30) to proximal (20) direction showing the respective slits (140′, 140″, 140″) together forming the passageway (150). Each and every slit (140′, 140″, 140″) is in mutual contact at a common slit crossing (152).

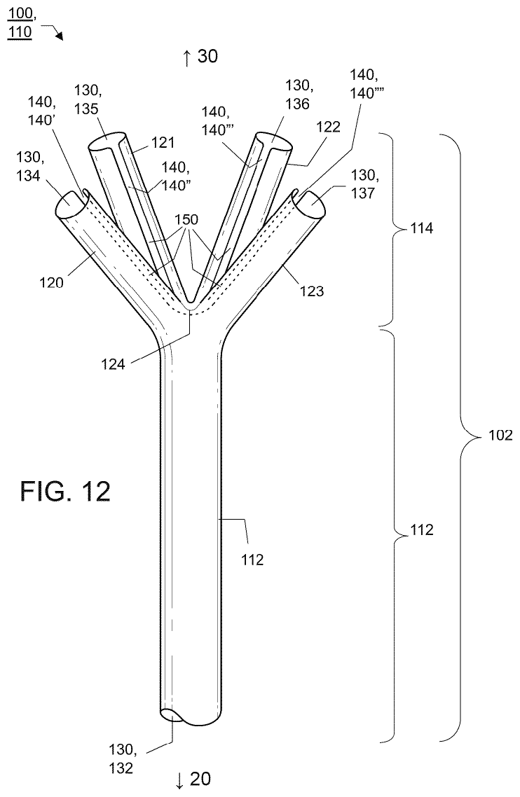

FIG. 12 depicts a delivery catheter (110) in an open configuration having a proximal (20) and distal end (30) comprising a first tube (102) disposed with a first lumen (130, 134, 135, 136, 137). The first tube (102) is furcated, namely quadfurcated at the distal end (30) at a furcation point (124), forming a proximal main part (112) disposed with the first lumen (132) and furcated part (114) where the first lumen (130, 134, 135, 136, 137) advances into each of the limbs (120, 121, 122, 123) of the furcated part (114). The furcated part (114) is disposed with a passageway (150) formed from a joining of longitudinal slits (140′, 140″, 140″, 140″) in each limb (120, 121, 122, 123) for passage of a quadfurcated stent.

FIG. 12A illustrates a view of FIG. 12 in a distal (30) to proximal (20) direction showing the together with the respective slits (140′, 140″, 140″, 140″) together forming the passageway (150). Each and every slit (140′, 140″, 140″, 140″) is in mutual contact at a common slit crossing (152).

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both, unless the disclosure states otherwise. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A delivery system for a furcated stent having a stem and at least two arms, the delivery system comprising:
a delivery catheter comprising an elongated first tube having a proximal end and a distal end, and a tubular furcated part at the distal end configured to accommodate the arms;
wherein:
tubular furcated part includes two or more limbs, each limb provided with a slit, thereby providing multiple limbs having multiple slits;
the multiple slits together form a passageway for passage therethrough of the furcated stent;
the passageway has a narrowed state and a widened state, is biased in the narrowed state, and is configured for repeatable transition between the narrowed state and the widened state.

2. The delivery system according to claim 1, wherein a quantity of arms of the furcated stent is 2, 3, 4, 5 or 6, and wherein a quantity of limbs of the tubular furcated part is 2, 3, 4, 5 or 6 respectively.

3. The delivery system according to claim 1, wherein the repeatable transition between the narrowed state and widened state of the passageway is actuated by application and/or withdrawal of mechanical force between the passageway and the furcated stent.

4. The delivery system according to claim 1, wherein the tubular furcated part is configured to constrain radially the arms when the passageway in the narrowed state and resist expansion of the furcated stent when the passageway in the narrowed state.

5. The delivery system according to claim 1, wherein each slit extends from each limb in a proximal direction from a distal terminal end of the limb, and connects at its proximal end to at least one other slit to form the passageway.

6. The delivery system of claim 1, wherein the slit has edges that are always detached from each other.

7. The delivery assembly of claim 1, wherein the multiple limbs are joined at a joint and spread apart as the limbs extend in a distal direction.

8. The delivery assembly of claim 1, wherein the furcated stent is self-expanding or non-self-expanding.

9. The delivery assembly of claim 1, wherein the tubular furcated part is formed of a compliant material that provides the bias of the passageway in the narrowed state and the repeatable transition between the narrowed state and widened state.

10. The delivery system according to claim 1, further comprising:
an access catheter including an elongated second tube having a proximal end region and a distal end region provided with a second lumen adapted to slidably accommodate the first tube, and configured to control a gradual opening or folding of the tubular furcated part of the first tube responsive to slidable relative displacement of the first and second tubes.

11. The delivery system according to claim 10, wherein the delivery catheter is configured for an over-the-wire or rapid exchange mode of operation.

12. The delivery system according to claim 10, further comprising the furcated stent that is self-expanding or non-self-expanding.

13. The delivery system according to claim 12, wherein the furcated stent is provided with an elutable active pharmaceutical ingredient.

14. The delivery system according to claim 12, wherein the furcated stent is prepared by laser cutting or braiding.

15. The delivery system according to claim 1, further comprising a pusher including an elongated flexible rod having a proximal end region and a distal end region, and a capture element at the distal end region of the elongated flexible rod for releasable attachment to the furcated stent at the proximal end region which and wherein the capture element is radially self-expanding to adopt an open or folded configuration,
wherein the folded configuration is configured for passage within a first lumen of the first tube and peripheral edges of the capture element are closer together to grip a proximal end of the furcated stent, and wherein the open configuration is configured for release of the furcated stent.

16. The delivery system according to claim 1, further comprising a loader for loading of the furcated stent into the delivery catheter comprising an elongated third tube having a proximal end and a distal end provided with a third lumen adapted to slidably accommodate the furcated stent in a folded configuration, wherein the distal end of the third tube is configured to couple with a proximal end of the delivery catheter such that a first lumen of the delivery catheter and the third lumen are connected to form a continuous passage for advancement of the furcated stent in the folded configuration from the loader to the delivery catheter.

17. The delivery system according to claim 1, wherein the delivery catheter is for a bodily vessel.

18. The delivery system according to claim 17, wherein the bodily vessel is an artery, a vein, a trachea-bronchial tree, a pancreaticobiliary duct, a urinary tract, salivary excretory duct.

19. A kit comprising the delivery catheter as defined in claim 1, further comprising at least one of: an access catheter, a pusher, a loader, or at least one guidewire.

20. A delivery assembly comprising:
a furcated stent including a stem and first arms;
a delivery catheter comprising an elongated first tube having a proximal end, and a distal end, and a tubular furcated part proximate the distal end of the first tube, wherein the tubular furcated part includes second tubular arms configured to receive within the second tubular arms the first arms of the furcated stent; and
each second tubular arm includes a slit extending substantially the length of the tubular second tubular arm, each slit contacting at least one other slit thereby forming a passageway, wherein the tubular furcated part is formed of a material having a native state that both applies a bias to the furcated stent resisting expansion of the furcated stent and allows forward and reverse passage of the furcated stent through the passageway, wherein the passageway has:
a narrowed state during which the second tubular arms extend circumferentially around a majority of the circumference of each of the first arms, and wherein the second tubular arms are biased in the narrowed state, and
a widened state formed by first arms forcing the passageway apart and the second arms extending circumferentially each of the first arms less than in the narrowed state.
